SEQUENCE LISTING (2 sequences)

SEQ ID NO: 1 - DNA, 20 bp, Artificial
  PCR primer
tttactgtggtagatactac

SEQ ID NO: 2 - DNA, 24 bp, Artificial
  PCR primer
gaaaaataaactgtaaatcatatt

(19) United States
(12) Patent Application Publication
(10) Pub. No.: US 2007/0026400 A1
(43) Pub. Date: Feb. 1, 2007

(54) Multi-hybridization set for dna microarray related assay

(76) Inventors: Sung Whan An, Taejeon (KR); Chi Wang Voon, Taejeon (KR); Tae Jeong Oh, Taejeon (KR); Dae Kyoung Yoon, Taejeon (KR); Sun Woo Lee, Taejeon (KR); Myung Soon Kim, Taejeon (KR); Suk Kyung Woo, Taejeon (KR)

Correspondence Address: INTELLECTUAL PROPERTY / TECHNOLOGY LAW, PO BOX 14329, RESEARCH TRIANGLE PARK, NC, 27709, US

(21) Appl. No.: 10/558,818

(22) PCT Filed: Jun. 7, 2003
(86) PCT No.: PCT/KR03/01115

§ 371 Date: Nov. 29, 2005

(51) Int. Cl. C12Q 1/68; C12M 1/34

(52) U.S. Cl. 435/6; 435/287.2

(57) ABSTRACT

The present invention relates to a multi-hybridization set for DNA microarray assay, which comprises: a lower plate in which a DNA microarray is mounted; an upper plate which is coupled to the lower plate, and in which one or more O-rings are attached to the lower surface of the upper plate coming in contact with the DNA microarray such that separate spaces are formed when the upper and lower plates are coupled to each other, the upper plate having sample injection holes formed inside of the O-ring; and means for coupling the upper and lower plates to each other. The multi-hybridization set of the present invention allows the DNA microarray assay of several samples to be performed at once. In addition, according to this multi-hybridization set, the problem of contamination caused by oil and dust, which occurs due to the use of the existing cover glass, can be solved.

### FIELD OF THE INVENTION

The present invention relates to a multi-hybridization set for use in microarray assay, which allows the assay of several samples to be performed at once, and also to a hybridization chamber system receiving the multi-hybridization set.

### BACKGROUND ART

Generally, a microarray system is a glass slide on which numerous genes amplified by the PCR method are deposited by means of a robotic array, and this glass slide containing genes thereon is also called a DNA microarray. Recently, microarray assay based on hybridization is most frequently used in various applications.

Currently, the microarray assay of a gene of various organisms in addition to the human body is conduced by many researchers. Moreover, as a method for the diagnosis of various diseases using the DNA microarray is developed, various diagnostic DNA microarrays are constructed for use.

The microarray assay is a process wherein DNA is deposited on a glass slide coated with specific chemicals making DNA well adhere to the microarray. Then, the deposited DNA is hybridized with a Cy3- or Cy5dUIP-labeled cDNA probe, and analyzed with a scanner. In this microarray assay process, the step of hybridizing genes (DNA or oligonucleotide) on the glass slide with the Cy3dUP or Cy5dUIP-labeled cDNA probe is highly critical.

The step of hybridizing the genes with the probe comprises dropping 20-40 μl of the probe onto the lass slide on which the gene was deposited; covering the glass slide with a cover slide; hybridizing the gene with the probe in a water bath at 50-65° C. for 2-8 hours according to a purpose; placing the glass slide on a slide rack; and washing the glass slide. However, in the case of currently used DNA microarrays, only one kind of DNA is deposited on one glass slide such that one kind of a sample can be analyzed. Thus, there is a limitation on the use of expensive DNA microarrays, and particularly, in the case of diagnostic microarrays, there is a problem in that they require larger costs than the prior PCR-based diagnostic method.

Furthermore, in general DNA microarray assay, a sample solution to be reacted puts on a DNA microarray and covered with a cover glass, and the resulting microarray puts in a hybridization chamber and then is subjected to hybridization. However, in this process, the cover glass contains oil and dust, etc., even if it is the best commercialized cover glass. For this reason, there are problems in that the assay often fails, and also an analyzer is liable to make a mistake during the step of covering the sample solution with the cover glass by means of tweezers, thereby reducing the reliability of assay results.

### DISCLOSURE OF THE INVENTION

Accordingly, the present inventors have attempted to solve the above-mentioned problems occurring in the prior art, and consequently, developed a multi-hybridization set, which allows various DNA samples to be deposited on one DNA microarray, such that a large number of reliable results can be obtained by one microarray assay. Furthermore, the present inventors have found that the application of the developed multi-hybridization set in hybridization analysis using a DNA microarray allowed DNA microassay assay to be performed in a more economical and efficient manner.

Therefore, an object of the present invention is to provide a multi-hybridization set, which comprises: a lower plate in which a DNA microarray is mounted; and an upper plate which is coupled to the lower plate, and in which one or more O-rings are attached to the lower surface of the upper plate coming in contact with the DNA microarray such that separate spaces are foamed when the upper and lower plates are coupled to each other, the upper plate having a sample injection hole inside each of the O-rings.

Another object of the present invention is to provide a hybridization chamber system, which comprises: a chamber which is capable of receiving a plurality of the multi-hybridization sets and in which a plurality of circular grooves are formed such that samples are prevented from being dried in a water bath; and a chamber lid.

To achieve the above objects, in one aspect, the present invention provides a multi-hybridization set for DNA microarray assay, which comprises: a lower plate in which a DNA microaray is mounted; an upper plate which is coupled to the lower plate, and in which one or more O-rings are attached to the lower surface of the upper plate coming in contact with the DNA microarray such that separate spaces are formed when the upper and lower plates are coupled to each other, the upper plate having a sample injection hole formed inside each of the O-rings; and means for coupling the upper and lower plates to each other.

Preferably, the number of the O-rings attached to the lower surface of the upper plate is 4-16 or 96, and the sample injection holes are formed by perforating the upper plate along the inner circumferential edge of each of the O-rings. More preferably, the number of the O-rings attached to the lower surface of the upper plate is 1-8, and the sample injection holes are formed by making a plurality of small holes in the inside of the respective O-rings.

Preferably, the multi-hybridization set according to the present invention has bolt or screw groove (or holes) foamed at the lower plate, and is provided with bolt or screw holes formed at the upper plate m such a manner as to correspond to the grooves (or holes) of the lower plate, so that the upper and lower plates are coupled to each other by means of bolts or screws.

In the multi-hybridization on set of the present invention, it is preferred that a pinhole is made in the upper plate and a pin is inserted into the pinhole, such that the upper and lower plates are easily separate from each other. Preferably, positioning protrusions are formed on the inner surface of either of the upper and lower plates, and positioning grooves are formed on the inner surface of the other plate in such a manner as to correspond to the positioning protrusions.

In another aspect, the present invention provides a hybridization chamber system comprising; a chamber which is capable of receiving a plurality of multi-hybridization sets and in which a plurality of circular grooves serving to prevent samples from being dried in a water bath are formed, and an O-ring for sealing with a lid of the chamber is formed at the inner circumference of the chamber, a lid of the chamber, and means for coupling the chamber lid to the chamber.

Preferably, the hybridization chamber system of the present invention additionally comprises dishes inserted into the grooves of the chamber and containing a drying-preventing agent, and the lid is provided with handles.

In the hybridization chamber system of the present invention, it is preferred that bolt or screw grooves (or holes) are formed on the chamber, and bolt or screw holes are formed in the lid in such a manner as to correspond to the grooves (or holes) of the chamber, such that the chamber and the chamber lid can be coupled to each other by means of bolts or a screws.

In a first preferred embodiment of the present invention, a multi-hybridization set 1 is constructed in such a manner as to be suitable for DNA microarray assay using the existing glass slide that is 7.5 cm wide and 2.5 cm long. An upper plate 10 is made of an acryl or aluminum sheet, which is 12.5 cm wide, 5.5 cm long and 1.0 cm. 4 to 20 O-rings are attached to the lower surface of the upper plate 10, and sample injection holes 11 are made in the inner circumferential edge of the respective O-ring. The size of the O-rings 14 attached to the lower surface of the upper plate may vary depending the number and shape of separate spaces to be formed in the multi-hybridization set.

In other words, when the separate spaces are 4 in number, they have an oval shape 11a as shown m FIG. 3, and are 2.2 cm wide and 1.2 cm long. When the separate spaces are 6-8 in number; they are circular, the diameter of the O-rings is about 1.0 cm, and the diameter of the holes formed inside the O-rings is 0.7 cm. If 10 to 20 O-rings are attached to the lower surface of the upper plate 10, their size needs to be made smaller and thus the size of the holes must also be reduced. The number of the O-rings attached to the lower surface of the upper plate 10 is preferably 1-20, more preferably 4-16, and most preferably 8.

If the O-rings attached to the upper plate 10 exceed 20 in number; the size of the holes 11 needs to be made smaller, and thus, it will be inconvenient to inject sample solutions in assay using the existing DNA microarray.

The O-rings 14 attached to the lower surface of the upper plate 10 are preferably made of rubber or silicon, and such rubber or silicon rings 14 come in close contact with a DNA microarray of the lower plate 20 such that a plurality of separate spaces are formed (see, FIGS. 1 and 2).

In order to make it easy to separate the upper plate from the lower plate after assay, a separate small hole 16 with a 0.5 cm diameter is made in the upper plate 10, and a plastic pin 15 is inserted into the hole 16.

Preferably, the lower plate 20 is made of an aluminum or acryl sheet that is 12.5 cm wide, 5.5 cm long and 0.8 cm high, and in the central portion of the lower plate 20, there is formed a groove 21 (8.0 cm wide and 2.5 cm long) capable of receiving DNA microarrays. In this case, in order to make it easy to separate the upper plate 10 from the lower plate 20 by the pin 15, the groove 21 of the lower plate 20 preferably comprises a deeper groove 24 extending from a portion coming in contact with the center of the bottom of the pin 15 to the end of the groove 21 adjacent thereto.

In order to connect the upper plate 10 to the lower plate 20, it is preferred that two bolt or screw holes 13 are made in the upper plate 10, and two bolt or screw grooves 23 are formed at the lower plate 20, so hat the upper 10 and lower 20 plates are coupled to each other by means of two bolts or screws 17 with a diameter of 2.5 cm.

Furthermore, in order to make it easy to couple the upper plate 10 to the lower plate 20, positioning protrusions 22 are formed on the inner surface of one of the upper 10 and lower 20 plate, positioning grooves 12 are formed on the other plate in such a manner as to correspond to the positioning protrusions 22. Preferably, the positioning grooves 12 are formed in a cylindrical shape with a diameter of 0.4 cm on the lower surface of the upper plate 10 in such a manner as to be diagonally arranged, and the protrusions 22, which are inserted into the grooves 12, are formed on the upper surface of the lower plate 20 to a height of 0.4 cm in such a manner as to be diagonally arranged.

In a multi-hybridization set according to a second preferred embodiment of the present invention, one to eight O-rings 14 are attached to the lower surface of the upper plate 10 coming in contact with DNA microarrays received in the lower plate, such that separate spaces are formed when the upper and lower plates are coupled to each other. Furthermore, a plurality of sample injection holes 45 with a diameter of 0.3-0.5 cm are made inside the respective O-rings other than making a hole along the inner circumferential edge of the respective O-rings (see, FIG. 4). The sample injection holes 45 are preferably 1 to 4 in number. The construction of the remaining elements is the same as the first embodiment.

Preferably, as shown m FIG. 4A, an oval O-ring 14, which is 6 cm wide and 2.2 cm long, is attached to the lower surface of the upper plate, and two sample injection holes 45 are formed side the O-ring in the upper plate 10. Alternatively, as shown in FIG. 4B, two O-rings 14 each having a 2.8 cm width and a 2.2 cm length are attached to the lower surface of the upper plate 10, and two sample injection holes 45 are formed inside the respective O-rings in the upper plate 10. Alternatively, as shown in FIG. 4C, two O-rings 14 each having a 1.2 cm width and a 2.2 cm length are attached to the lower surface of the upper plate 10, and two sample injection holes 45 are formed inside the respective O-rings in the upper plate 10.

A chamber 40 capable of receiving a plurality of the multi-hybridization sets according to the first or second embodiment of the present invention and performing hybridization in a water bath is preferably 250 cm wide, 175 cm long and 5 cm high, and made of aluminum or acryl (see, FIG. 6). Furthermore, the chamber lid 30 preferably provided with handles 32 on both sides thereof and made of an aluminum sheet with a thickness of 0.5 cm.

In the chamber lid 30 and the chamber 40 constructed as described above, it is preferred that six bolt or screw holes are formed in the chamber lid 30, and bolt or screw grooves 43 are formed on the chamber 40, so that the chamber lid 30 and the chamber 40 are coupled to each other by means of six bolts or screws 31.

On the bottom 44 of the chamber 40, two circular grooves 41 of a 4.0 cm diameter each receiving a plastic dish 42 containing 3×SSC are preferably formed, but not limited to two in number. Such grooves act to prevent the drying of samples by the maintenance of humidity when the chamber 40 is kept in a water bath at 50-65° C. for 2-8 hours for hybridization.

Although the present invention illustrates that four multi-hybridization sets 1 are received in the chamber 40, it will be obvious to a person skilled in the art that the number of the multi-hybridization sets, which are received in the chamber, may be increased or reduced according to circumstances.

In a third preferred embodiment of the present invention, a multi-hybridization set is constructed in such a manner as to be suitable for assay using the existing 96-well DNA microarray (see, FIG. 5). 96 (12×8) holes are made in an upper plate having a 14 cm width, a 9 cm length and a 1.0 cm height, and an O-ring is attached to the lower surface of the upper plate 10 at the outer circumferential edge of the respective holes. A lower plate 20 is the same size as the upper plate 10, and in order to make it easy to separate the upper plate 10 from the lower plate 20, two separate small holes 16 with a diameter of 0.5 cm are made in the upper plate 10, and plastic pins 15 are inserted into the holes 16.

Preferably, the lower plate 20 is made of an aluminum sheet having a 14 cm width, a 9 cm length and a 0.8 cm thickness, and at the central portion thereof, there is formed a groove (12.5 cm wide and 8.5 cm long) 21 capable of receiving DNA microarrays.

Furthermore, in order to make it easy to couple the upper plate 10 to the lower plate 20, positioning protrusions 22 are formed on the inner surface of one of the upper 10 and lower 20 plates, and positioning grooves 12 are formed on the inner surface of the other plate in such a manner as to respond to the positioning protrusions 22. Preferably, the grooves 12 are formed in a cylindrical shape with a diameter of 0.4 cm on the lower surface of the upper plate 10, and the protrusions 22, which are inserted into the grooves 12, are formed on the upper sur of the lower plate 20 to a height of 0.4 cm. In addition, in order to couple the upper plate 10 to the lower plate 20, it is preferred that four bolt or screw holes 13 are made in the upper plate 10, and four bolt or screw grooves 23 are formed at the upper surface of the lower plate 20, so that the upper and lower plates are coupled to each other by means of four bolts or screws 17 with a diameter of 2.5 cm. The construction of the remaining elements is the same as the fist embodiment.

### BRIEF DESCRIPTION OF THE DRAWINGS

FIG. 1 shows a multi-hybridization set with eight holes according to a first embodiment of the present invention.

FIG. 2 shows a state where an upper plate and lower plate of a multi-hybridization set with eight holes were coupled to each other.

FIG. 3 shows an upper plate of a multi-hybridization set with four holes according to a first embodiment of the present invention.

FIGS. 4A to 4C show an upper plate of a multi-hybridization set according to a second embodiment of the present invention, m which a plurality of sample injection holes are formed in the upper plate at one separate space defined by one O-ring. FIG. 4A shows an embodiment where one O-ring is attached to the lower surface of the upper plate, and two sample injection holes are made in the upper portion at the inside of the O-ring. FIG. 4B shows an embodiment where two O-rings are attached to the lower surface of the upper plate, and two sample injection holes are made in the upper plate at the inside each of the O-rings. FIG. 4C shows an embodiment where four O-rings are attached to the lower surface of the upper plate, and two sample injection holes are made in the upper plate at the inside each of the O-rings.

FIG. 5 shows a multi-hybridization set with 96 holes according to a bird embodiment of the present invention.

FIG. 6 shows a hybridization chamber system receiving multi-hybridization sets.

FIG. 7 shows the result of DNA microarray assay using a multi-hybridization set having 8 holes at an upper plate.

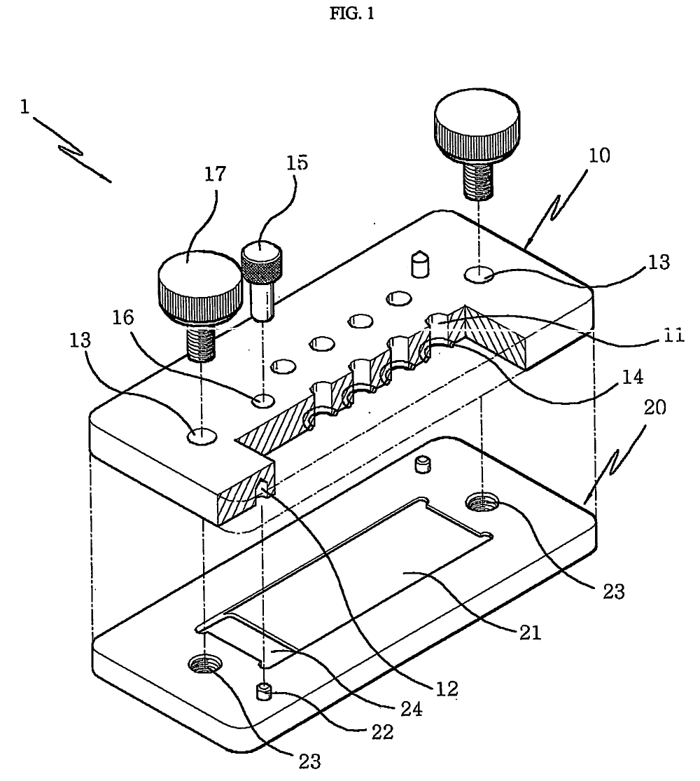

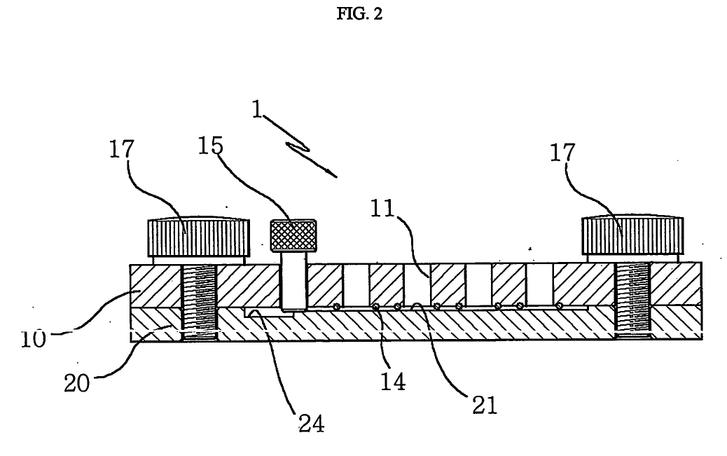

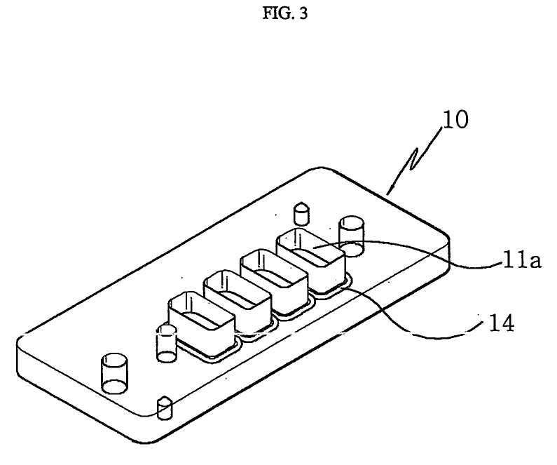

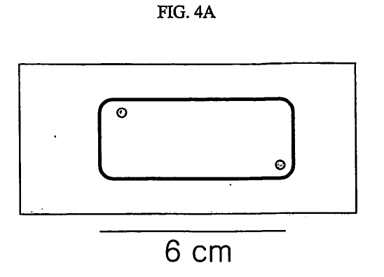

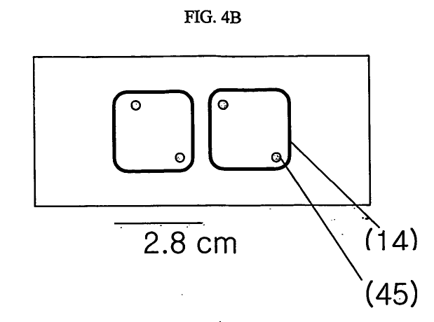

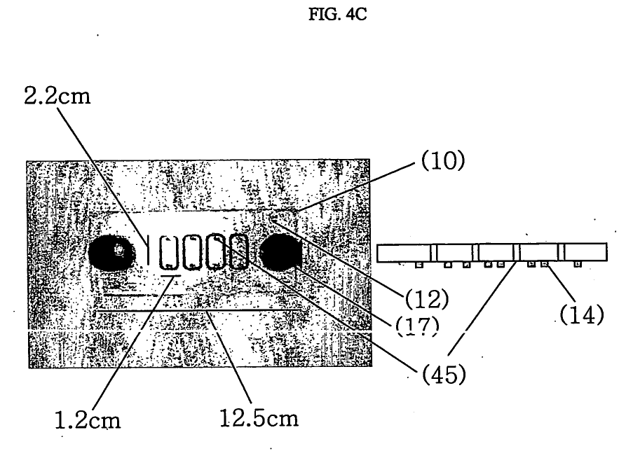

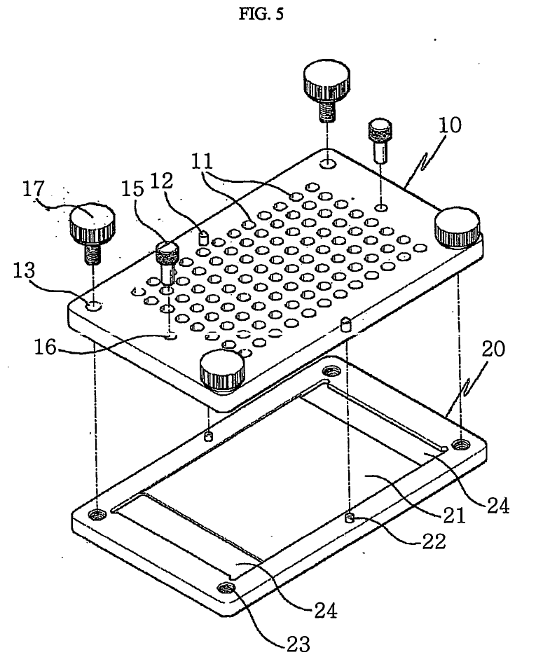

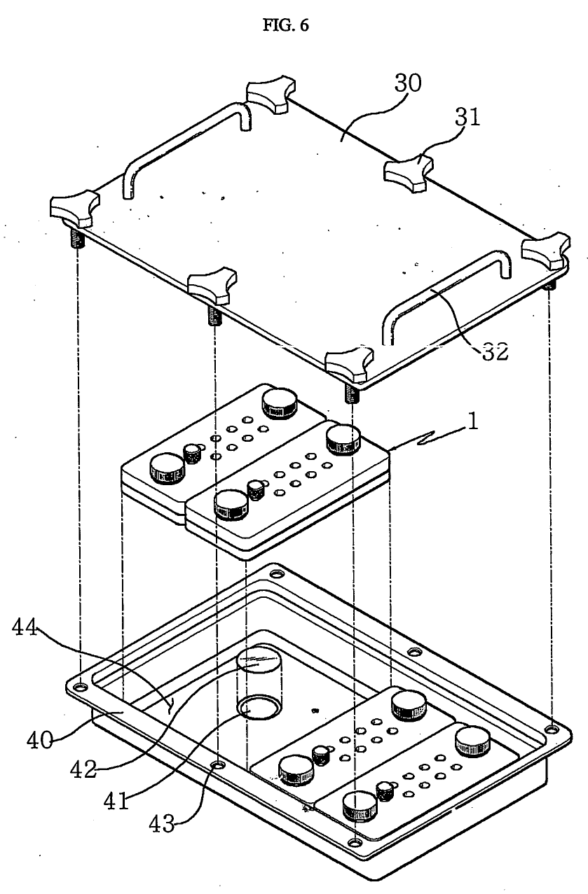

### DETAILED DESCRIPTION OF THE INVENTION

The present invention will hereinafter be described in further detail by examples. It should however be borne in mind that these examples are intended to illustrate DNA microarry assay using a multi-hybridization set and hybridization chamber system of the present invention, and the scope of the present invention is not limited to or by the examples.

### EXAMPLE

A glass slide, which had been coated with chemicals making DNA adheres well to the glass slide, such as silane, amine, aldehyde or poly-L-lysine, put on a microarray for diagnosis or study, and then DNA, cDNA or oligo-DNA samples were deposited on the glass slide at eight fixed spots. Next in order to test the multi-hybridization set and hybridization chamber system of the present invention through the steps of isolation of DNA from the samples, labeling of DNA with a flurescent substance, washing and scanning, DNA microarray assay for the diagnosis of HPV (human papilloma virus) was carried out as follows.

In order to confirm if the multi-hybridization set and hybridization chamber system of the present invention are suitable for use in HPV typing, genomic DNA was isolated from three human cell lines (Caski (HPV 16), Hela (HPV18), and SiHa (HPV 16)) known to be already infected with HPV. Then, the target DNA was labeled with a fluorescent substance of Cy3-dUIP (cyanine-3dUIP) by a PCR method using a primer of GP5+(5′TTTACTGTGGTAGATACTAC-3′ (SEQ ID NO: 1)/GP6+(5′GAAAAATAAACTGTAAATCATATT-3′) (SEQ ID NO: 2). In the PCR reaction, 50 μl of reaction solution comprising 10× PCR buffer (Solgent Co.), 5 μl of 2.5 mM dNIP (deoxynucleotide phosphate), 100 ng of genomic DNA, 1 μl of 25 nM Cy3-dUIP (deoxy uracil phosphate) and 2.5 units of taq polymerase (Solgent Co.) were used, and the PCR reaction consisted of: initial denaturation at 94° C. for 10 minutes; 30 cycles of denaturation at 94° C. for 1 minute, annealing at 55° C. for 45 seconds, and extension at 72° C. for 45 seconds; and final extension at 72° C. for 10 minutes. The PCR products labeled with Cy3 were purified with a QuiaQuia PCR purification kit and concentrated to a final volume of 25 μl. These DNA solutions were hybridized with an HPV DNA microarray This hybridization was carried out at 50° C. for 2 hours using a hybridization solution composing 3×SSC, 0.2% SDS and 20 μg salmon sperm DNA for a 40 μl final volume.

For this hybridization reaction, a DNA microarry put on the lower plate of the multi-hybridization set, and was covered with the upper plate according to the second embodiment of the present invention, and then the upper and lower plates were coupled to each other by means of bolts or screws. Next, the hybridization solutions (samples solutions) were injected into holes formed in the upper plate. The sample solutions were maintained m the separate spaces defined by the rubber or silicon rings located below the respective holes without leakage. Then, the multi-hybridization set put in the chamber of the hybridization chamber system, and was covered with the chamber lid. Also, in order to maintain the humidity within the chamber, plastic dishes with a 40 cm diameter and a 7 cm height, which contain 3×SSC, were disposed within the chamber.

After hybridization, the microarray was washed with 2×SSC for 2 minutes, 0.2×SSC and 0.1% SDS for 5 minutes and 0.2×SSC for 5 minutes, and centrifuged at 700 rpm (Hanil Co.) for 10, and then dried. Then, the result of the hybridization reaction was analyzed using a scanner (Axon 4000B).

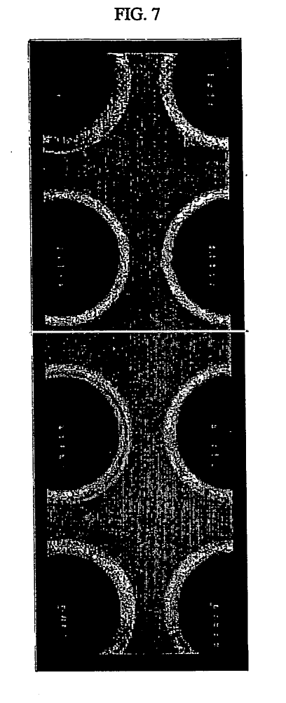

As a result of the HPV DNA microarray assay, it could be found that an A549 cell line uninfected with HPV was hybridized only at a globin gene as a PCR control, and the remaining cell lines (Caski, Hela and SiHa) were specifically hybridized only at an HPV E1 gene as a positive control and a cell type-specific DNA (see, FIG. 7).

### INDUSTRIAL APPLICABILITY

As described above, the multi-hybridization set according to the present invention allows a plurality of DNA probes to be deposited on one DNA microarray in DNA microarray assay such that several samples can be assayed at once. This provides a reduction in time and costs. Furthermore, the multi-hybridization set of the present invention has an effect in that it does not require the use of a cover glass and thus reduces the failure rate of assay. In addition, there is an effect in that the samples are prevented from being dried in a water bath, such that reliable assay results can be obtained.

1. A multi-hybridization set for DNA microarray assay, which comprises:
a lower plate in which a DNA microaray is mounted;
an upper plate which is coupled to the lower plate, and in which one or more O-rings are attached to the lower surface of the upper plate coming in contact with the DNA microarray such that separate spaces are formed when the upper and lower plates are coupled to each other, the upper plate having sample injection holes formed inside of the O-ring; and
means for coupling the upper and lower plates to each other.

2. The multi-hybridization set of claim 1, wherein bolt or screw grooves (or holes) are formed on the upper surface of the lower plate, and bolt or screw holes are made in the upper plate in such a manner as to correspond to the grooves (or holes) of the lower plate, such that the upper and lower plates can be coupled to each other by means of bolts or screws.

3. The multi-hybridization set of claim 1, wherein a pinhole is made in the upper plate and a pin is inserted into the pinhole, such that the upper and lower plates are easily separated from each other.

4. The multi-hybridization set of claim 1, wherein positioning protrusions are formed on the inner surface of either of the upper or lower plates, and positioning grooves are formed on the inner surface of the other plate in such a manner as to correspond to the positioning protrusions.

5. The multi-hybridization set of claim 1, wherein the number of the O-rings attached to the lower surface of the upper plate is 4 to 16, and the sample injection holes are formed by perforating the upper plate along the inner circumferential edge of each of the O-rings.

6. The multi-hybridization set of claim 1, wherein the number of the O-rings attached to the lower surface of the upper plate is 1 to 8, and a plurality of the sample injection holes are formed at the inside each of the O-rings.

7. The multi-hybridization set of claim 1, wherein the number of the O-rings attached to the lower surface of the upper plate is 9×6, and the sample injection holes are formed by perforating the upper plate along the inner circumferential edge of each of the O-rings.

8. A hybridization chamber system comprising:
a chamber which is capable of receiving a plurality of multi-hybridization sets according to claim 1 and in which a plurality of circular grooves serving to prevent samples from being dried in a water bath are formed on the bottom of the chamber, and an O-ring for sealing with a lid of the chamber is formed at the inner circumference of the chamber;
a lid of the chamber; and
means for coupling the chamber and the chamber lid to each other.

9. The hybridization chamber system of claim 8, which additionally comprises dishes inserted into the grooves of the chamber and containing a drying-preventing agent.

10. The hybridization chamber system of claim 8, wherein bolt or screw grooves (or holes) are formed at the chamber, and bolt or screw holes are formed at the lid in such a manner as to correspond to the grooves (or holes) of the chamber, such that the chamber and the chamber lid can be coupled to each other by means of bolts or screws.

11. The hybridization chamber system of claim 8, wherein the chamber lid is provided with handles.

12. The hybridization chamber system of claim 8, wherein in said multi-hybridization sets, bolt or screw grooves (or holes) are formed on the upper surface of the lower plate, and bolt or screw holes are made in the upper plate in such a manner as to correspond to the grooves (or holes) of the lower plate, such that the upper and lower plates can be coupled to each other by means of bolts or screws.

13. The hybridization chamber system of claim 8, wherein in said multi-hybridization sets, a pinhole is made in the upper plate and a pin is inserted into the pinhole, such that the upper and lower plates are easily separated from each other.

14. The hybridization chamber system of claim 8, wherein in said multi-hybridization sets, positioning protrusions are formed on the inner surface of either of the upper or lower plates, and positioning grooves are formed on the inner surface of the other plate in such a manner as to correspond to the positioning protrusions.

15. The hybridization chamber system of claim 8, wherein in said multi-hybridization sets, the number of the O-rings attached to the lower surface of the upper plate is 4 to 16, and the sample injection holes are formed by perforating the upper plate along the inner circumferential edge of each of the O-rings.

16. The hybridization chamber system of claim 8, wherein in said multi-hybridization sets, the number of the O-rings attached to the lower surface of the upper plate is 1 to 8, and a plurality of the sample injection holes are formed at the inside each of the O-rings.

17. The multi-hybridization set of claim 1, wherein said means for coupling the upper and lower plates to each other comprise mechanical fasteners.

18. The multi-hybridization set of claim 1, wherein said means for coupling the upper and lower plates to each other comprise matably engageable groove and protrusion elements.

19. A multi-hybridization set for DNA microarray assay, which comprises:
a lower plate in which a DNA microarray is mounted; and
an upper plate which is coupled to the lower plate, and in which one or more O-rings are attached to the lower surface of the upper plate coming in contact with the DNA microarray such that separate spaces are formed when the upper and lower plates are coupled to each other, the upper plate having sample injection holes formed inside of the O-ring.

20. A multi-hybridization set for DNA microarray assay, which comprises:
a lower plate adapted for mounting of a DNA microaray therein; and
an upper plate which is coupled to the lower plate, and in which one or more O-rings are attached to a lower surface of the upper plate coming in contact with the DNA microarray such that separate spaces are formed when the upper and lower plates are coupled to each other, the upper plate having sample injection holes formed inside of the O-ring.
